(19) United States
(12) Patent Application Publication
(10) Pub. No.: US 2012/0022149 A1
(43) Pub. Date: Jan. 26, 2012

(54) PHARMACEUTICAL COMPOSITION WITH ENHANCED SOLUBILITY CHARACTERISTICS

(76) Inventors: Masood A. Chowhan, Arlington, TX (US); Malay Ghosh, Fort Worth, TX (US); Wesley Wehsin Han, Arlington, TX (US); Way-Yu Lin, Arlington, TX (US); Vincent Nguyen, Grapevine, TX (US)

(21) Appl. No.: 13/186,516

(22) Filed: Jul. 20, 2011

Related U.S. Application Data
(60) Provisional application No. 61/366,328, filed on Jul. 21, 2010.

(51) Int. Cl. A61K 31/335; A61P 27/02

(52) U.S. Cl. 514/450

(57) ABSTRACT

There are disclosed pharmaceutical compositions, particularly ophthalmic compositions, that contain relatively high concentrations of solubility enhancing polymer (e.g., polyether polymer, polyvinyl polymer or a combination thereof) for providing enhanced solubility of one or more therapeutic agents. In a preferred embodiment, the composition is a multi-dose topical aqueous ophthalmic composition that contains relatively high concentrations of solubility enhancing polymer (e.g., polyether polymer, polyvinyl polymer or a combination thereof) for providing enhanced solubility of one or more therapeutic agents.

### CROSS-REFERENCE TO RELATED APPLICATION

The present application claims priority based on U.S. Provisional Patent Application Ser. No. 61/366,328 filed Jul. 21, 2010.

### TECHNICAL FIELD OF THE INVENTION

The present invention is related to pharmaceutical composition (e.g., ophthalmic compositions) that contain relatively high concentrations of solubility enhancing polymer (e.g., polyether polymer, polyvinyl polymer or a combination thereof) for providing enhanced solubility of one or more therapeutic agents. More specifically, the present invention relates to multi-dose topical aqueous ophthalmic compositions that contain relatively high concentrations of solubility enhancing polymer such as polyethylene glycol for providing enhanced solubility of one or more therapeutic agents.

### BACKGROUND OF THE INVENTION

Therapeutic agents that exhibit low solubility in water have been problematic to pharmaceutical industry in general and particularly problematic when forming aqueous ophthalmic compositions. The concentration of a therapeutic agent that can be solubilized in an aqueous composition can at least partially dictate the ability that the composition will have in providing the desired therapeutic effect. As a result, the amount of therapeutic agent that can be solubilized can also at least partially dictate the amount and/or frequency of dosing for an ophthalmic composition or other pharmaceutical composition.

It is particularly desirable to maintain a relatively low frequency of dosing for ophthalmic compositions since delivery of the compositions to the eye can be relatively inconvenient. For example, topical applications of ophthalmic compositions (e.g., eye drops) can be difficult to administer, particularly for the elderly, since they often require a high degree of manual dexterity and because it can be difficult to determine whether an eye drop was fully dispensed to the cornea of the eye. Such topical applications can also result in undesirable attention being drawn to the individual dosing the composition if such dosing must take place in a public place or may require an individual to take time out from activities to find a private place to provide dosing. Thus, low frequency dosing of compositions with higher solubilized concentrations of therapeutic agent are often preferred.

Many solubility issues can be addressed simply by providing one of many know surfactants or solubility enhancing agents to an ophthalmic composition to allow a sufficient concentration of therapeutic agent to be solubilized therein. However, the type of therapeutic agent, the desired concentration of therapeutic agents or other factors can give rise to solubility issues that cannot simply be addressed through the use of surfactants or they can require the use of undesirably high concentrations of surfactant. Finding solutions to such solubility issues can be extremely problematic.

For ophthalmic and other pharmaceutical compositions, the formulator of the composition not only needs to address the solubility issue, but will typically also need to address a host of other issues that can be brought about by attempts to increase therapeutic agent concentration. As one example, stability of a therapeutic agent can become more critical when a high concentration of therapeutic agent is employed. Larger amounts of unstable therapeutic agent will typically result in larger amounts of undesirable degradation products. As another example, the use of greater amounts of solubility agent may cause incompatibility with aqueous phase leading to an unstable product. Still further, and particularly for ophthalmic compositions, the use of greater amounts of solubility agent can cause an eye drop to be irritating to the eye.

In view of the above, it would be particularly desirable to provide a pharmaceutical composition, particularly an ophthalmic composition, that allows for the solubility of higher concentrations of relatively insoluble therapeutic agents while avoiding other drawbacks typically associated with such efforts.

### SUMMARY OF THE INVENTION

Accordingly, the present invention is directed to a multi-dose aqueous pharmaceutical composition that comprises a therapeutic agent, a solubility enhancing polymer and water. The therapeutic agent will typically exhibit relatively low solubility in water. The therapeutic agent is present and solubilized in the composition at a concentration that is at least 100% greater than a concentration of the therapeutic agent at a maximum solubility of the therapeutic agent in water. The solubility enhancing polymer is present in the composition at a concentration that is at least 5 w/v % but no greater than 50 w/v %. The solubility enhancing polymer is typically selected from a polyether polymer, a polyvinyl polymer or a combination thereof. The composition also typically includes at least 50 w/v % water and preferably a quantity sufficient of water to arrive at the desired concentrations of therapeutic agent and/or solubility enhancing polymer.

In a preferred embodiment, the solubility enhancing polymer includes polyethylene glycol and preferably includes at least 90% by weight polyethylene glycol. When included the polyethylene glycol preferably has a number average molecular weight that is at least 4000 but no greater than 8000 and even more preferably has a number average molecular weight that is at least 5000 but is no greater than 7000.

A preferred therapeutic agent is an anti-allergy medication. A highly preferred therapeutic agent is olopatadine.

The composition may further comprise a stabilizer selected from an anti-oxidant, a reducing agent or a combination thereof. A preferred stabilizer can be selected from sodium thiosulfate, sodium borohydride, sodium pyruvate and combinations thereof.

In a highly preferred embodiment, the composition is a multi-dose ophthalmic composition. As such, the composition may be disposed within an eyedropper.

The present invention is also directed to a method of administering an ophthalmic composition to the eye. The method typically includes application of the composition described above to the surface of the eyeball. More preferably the method includes application of the composition described above to the surface of the eyeball as one or more eyedrops from the eyedropper.

### BRIEF DESCRIPTION OF THE DRAWINGS

FIG. 1 is a graph of showing the solubility of Olopatadine relative to the concentration of different molecular weights of polyethylene glycol according to an aspect of the present invention.

### DETAILED DESCRIPTION OF THE INVENTION

The present invention is directed at the provision of a pharmaceutical composition having a relatively high concentration of therapeutic agent and a relatively high concentration of polymeric solubility enhancing agent while avoiding issues otherwise typically caused by such high concentrations. The composition may be an otic or nasal composition; however, it is preferably an ophthalmic composition. The therapeutic agent will typically be an agent having a relatively low solubility in water particularly at physiologic pH, which, for the present invention is considered to be 6.5 to 7.5. The present invention may also provide for enhanced stability of the therapeutic agent.

Unless otherwise indicated, percentages provided for the ingredients of the ophthalmic composition of the present invention are weight/volume (w/v) percentages.

The therapeutic agent of the present invention can include one or more different chemical entities. Moreover, the therapeutic agent of the present invention is typically one that exhibits a relatively low solubility in water. As such, the therapeutic agent typically has a log D that is greater than 0.1, more preferably greater than 0.4, more preferably greater than 0.6 and even possibly greater than 1.0 or even greater than 1.5.

As used herein, log D is the ratio of the sum of the concentrations of all forms of the therapeutic agent (ionized plus un-ionized) in each of two phases, an octanol phase and a water phase. For measurements of distribution coefficient, the pH of the aqueous phase is buffered to 7.4 such that the pH is not significantly perturbed by the introduction of the compound. The logarithm of the ratio of the sum of concentrations of the solute's various forms in one solvent, to the sum of the concentrations of its forms in the other solvent is called Log D:

log D_{oct/wat}=log([solute]_{octanol}/([solute]_{ionized water}+[solute]_{neutral water}))

Agents which may be suitable in the composition of the invention include anti-VEGF antibody (i.e., bevacizumab or ranibizumab); VEGF trap; siRNA molecules, or a mixture thereof, targeting at least two of the tyrosine kinase receptors having IC₅₀ values of less than 200 nM in Table 1; glucocorticoids (i.e., dexamethasone, fluoromethalone, medrysone, betamethasone, triamcinolone, triamcinolone acetonide, prednisone, prednisolone, hydrocortisone, rimexolone, and pharmaceutically acceptable salts thereof, prednicarbate, deflazacort, halomethasone, tixocortol, prednylidene (21-diethylaminoacetate), prednival, paramethasone, methylprednisolone, meprednisone, mazipredone, isoflupredone, halopredone acetate, halcinonide, formocortal, flurandrenolide, fluprednisolone, fluprednidine acetate, fluperolone acetate, fluocortolone, fluocortin butyl, fluocinonide, fluocinolone acetonide, flunisolide, flumethasone, fludrocortisone, fluclorinide, enoxolone, difluprednate, diflucortolone, diflorasone diacetate, desoximetasone (desoxymethasone), desonide, descinolone, cortivazol, corticosterone, cortisone, cloprednol, clocortolone, clobetasone, clobetasol, chloroprednisone, cafestol, budesonide, beclomethasone, amcinonide, allopregnane acetonide, alclometasone, 21-acetoxypregnenolone, tralonide, diflorasone acetate, deacylcortivazol, RU-26988, budesonide, and deacylcortivazol oxetanone); Naphthohydroquinone antibiotics (i.e., Rifamycin).

In a highly preferred embodiment, the therapeutic agent includes an ocular anti-allergy medication that is a mast cell stabilizer, an antihistamine or both. The most preferred anti-allergy medication is olopatadine, which, as referred to herein, includes any chemical entity having olopatadine such as a salt of olopatadine. Particularly preferred is olopatadine hydrochloride. Thus, in a preferred embodiment, the therapeutic agent consists essentially of or consists entirely of olopatadine. It has been found that the present invention is particularly desirable for forming compositions with high concentrations of ocular anti-allergy medication. Such compositions are particularly desirable since such medications, particularly olopatadine, can exhibit both early and late stage efficacy against ocular allergy when dosed once daily at a relatively high concentration.

The therapeutic agent is typically present in the composition in a solubilized concentration that is at least 0.1 w/v %, more typically at least 0.25 w/v %, still more typically at least 0.3 w/v % and even possibly at least 0.35 w/v % or even at least 0.5 w/v %. The therapeutic agent is also typically present in the composition in a solubilized concentration that is no greater than 4.0 w/v % and more typically no greater than 2.0 w/v %. As used herein, a solubilized concentration refers to the concentration of the drug in the composition that is actually solubilized. It is also typical for the therapeutic agent to be present and solubilized in the composition at a concentration that is at least 50% greater, more typically at least 100% greater and even more typically at least 150% or even 200% greater than a concentration of the therapeutic agent at a maximum solubility of the therapeutic agent in water alone. Thus, for an agent that exhibits a maximum concentration of solubilized agent in purified water alone of 1.0 w/v %, that agent can be present in a solubilized concentration in composition of the present invention that is at least 50% greater (i.e., 1.5 w/v % or more) or at least 100% greater (i.e., 2.0 w/v % or more) or at least 200% greater (i.e., 3.0 w/v % or more) when the composition of the present invention is brought to the same pH (e.g., through use of HCl or NaOH) as a solution containing the maximum concentration of solubilized agent in purified water. Typically, the therapeutic agent of the present invention exhibits a solubility in water of no greater than about 0.5%, more typically no greater than about 0.3% and even possibly no greater than about 0.22% or even no greater than about 0.2% at a pH of 7.0, atmospheric pressure and a temperature of 25° C.

The polymeric solubility enhancing agent can comprise one, two or more polymers. Polyvinyl polymer, polyether polymers or combinations thereof are particularly desirable for the present invention. At relatively high concentrations, these polymers can significantly aid in solubilizing the therapeutic agent. The polymeric solubility enhancing agent may consist or consist essentially of polyvinyl polymer but preferably includes a substantial amount of polyether polymer. In one preferred embodiment, the polymeric solubility enhancing agent consists or consists essentially of polyether polymer.

Polyvinylpyrrolidone (PVP) is a particularly preferred polyvinyl polymer. PVP can aid in solubilizing the therapeutic agent and/or stabilizing the therapeutic agent particularly when the therapeutic agent in olopatadine. Thus, the polyvinyl polymer of the composition of the present invention can consist or consist essentially of PVP. Polyvinylpyrrolidone is a known polymer and is commercially available from a variety of sources in different grades and in a number of molecular weights. For example, polyvinylpyrrolidone is available in many grades from International Specialty Products (Wayne, N.J.): Plasdone® C-15 (weight avg. MW=8K), K-26/28 (weight avg. MW=30K), K-29/32 (weight avg. MW=58K), K-30 (weight avg. MW=50K) and K-90 (weight avg. MW=1300K). Also, polyvinylpyrrolidone is available from BASF Corporation under the Kollidon brand name. As used herein, “polyvinylpyrrolidone” includes homopolymers of vinylpyrrolidone and copolymers of vinylpyrrolidone and vinyl acetate. Vinylpyrrolidone-vinyl acetate copolymers are known as “copovidone” and are commercially available from BASF Corporation as Kollidon VA 64. The polyvinylpyrrolidone ingredient included in the compositions of the present invention has a weight average molecular weight of 5000-1,600,000. Most preferred is polyvinylpyrrolidone having a weight average molecular weight of 50,000-60,000. In general, the amount of polyvinylpyrrolidone contained in the compositions of the present invention will be 0.1-3%, preferably 0.2-2%, and most preferably 1.5-2%. Advantageously, PVP can have a stabilizing effect on therapeutic agent as well as a solubilizing effect. This is particularly the case for olopatadine.

Polyethylene glycol (PEG) is a particularly preferred polyether polymer for the present invention. Thus, the polyether polymer of the composition of the present invention can consist or consist essentially of PEG. Like PVP, PEG is a known polymer that is available from a variety of different sources and can have a variety of different molecular weights. As used herein polyethylene glycol can include homopolymers of PEG and copolymers including PEG. In a preferred embodiment, the PEG is at least 90% by weight, more typically at least 97% by weight and even possibly entirely homopolymers of PEG. The concentration of PEG in the composition will typically be at least 5 w/v %, more typically at least 10 w/v %, even more typically at least 15 w/v % and even possibly at least 20 w/v % or even at least 25 w/v %. The concentration of PEG in the composition will typically be no greater than 50 w/v % and even more typically no greater than 40 w/v % or even no greater than 30 w/v %. It has been found that, since a relatively large concentration of PEG can be employed in the composition, the molecular weight of that PEG can be very important in producing a desirable ophthalmic composition. If the molecular weight of the PEG is too low, the relatively high concentration of PEG can raise the osmolality of the composition to levels that can irritate the eye. Thus, the molecular weight of the PEG is typically at least 1000, more typically at least 3000 and even more typically at least 4000 or even at least 5000. If the molecular weight of the PEG is too high, the composition can become too viscous and unsuitable for dispensing. Thus, the molecular weight of the PEG is typically no greater than 12000, more typically no greater than 9000 and still more typically no greater than 8000 or even no greater than 7000. As used herein the molecular weight of PEG is taken as a number average molecular weight. Advantageously, it has been found that relatively high amounts of PEG can be employed without sacrificing comfort, particularly ocular comfort.

It is preferable, although not required unless otherwise specifically stated, for the ophthalmic composition of the present invention, depending upon the therapeutic agent in the composition, to include a stabilizer. A variety of stabilizers known in the art can be included. Suitable examples include, without limitation, anti-oxidants, reducing agents, oxidizing agents, free radical scavengers, any combinations thereof or the like. Generally, when used, the stabilizer, depending upon its type can be present in the composition in a concentration that is at least 0.0001 w/v % and more preferably at least 0.001 w/v % and even possibly at least 0.1 w/v %, but that is typically no greater than 10 w/v %, more typically no greater than 1 w/v % and even possibly no greater than 0.5 w/v %.

In addition to or as an alternative to the stabilizers above, other stabilizers have been found to be particularly useful with olopatadine when used in conjunction with the present invention. Generally those stabilizers are either antioxidants or reducing agents. Examples of highly preferred antioxidants for use in conjunction with olopatadine agents are sodium thiosulfate, sodium pyruvate or a combination thereof. An example of a suitable reducing agent suitable for use in conjunction with olopatadine is sodium borohydride. It is also contemplated that any combination of these named anti-oxidants or the named reducing agent may be used according to the present invention. While these particular antioxidants and reducing agents have been found particularly desirable for use in conjunction with olopatadine, it is contemplated that they may also be employed in conjunction with other therapeutic agents as well. When antioxidant, reducing agent or a combination thereof are employed as the stabilizer, the stabilizer can be present in the composition in a concentration that is at least 0.0001 w/v % and more preferably at least 0.005 w/v % and even possibly at least 0.01 w/v %, but that is typically no greater than 1 w/v %, more typically no greater than 0.1 w/v % and even possibly no greater than 0.05 w/v %.

For topical application, the compositions of the present invention typically include antimicrobial agent. Potential antimicrobial agents include, without limitation, hydrogen peroxide, chlorine containing preservatives such as benzalkonium chloride, biguanides, polymeric quaternary ammonium compound or others.

The composition and/or vehicle of the present invention can also include an antimicrobial buffer system such as a borate/polyol complex system. An example of one potentially suitable system is discussed in U.S. Pat. No. 6,143,799, which is incorporated herein by reference for all purposes.

As used herein, the term “borate” shall refer to boric acid, salts of boric acid, borate derivatives and other pharmaceutically acceptable borates, or combinations thereof. Most suitable are: boric acid, sodium borate, potassium borate, calcium borate, magnesium borate, manganese borate, and other such borate salts. Borate interacts with polyols, such as glycerol, propylene glycol, sorbitol and mannitol, to form borate polyol complexes. The type and ratio of such complexes depends on the number of OH groups of a polyol on adjacent carbon atoms that are not in trans configuration relative to each other. It shall be understood that weight/volume percentages of the ingredients polyol and borate include those amounts whether as part of a complex or not.

As used herein, the term “polyol” includes any compound having at least one hydroxyl group on each of two adjacent carbon atoms that are not in trans configuration relative to each other. The polyols can be linear or cyclic, substituted or unsubstituted, or mixtures thereof, so long as the resultant complex is water soluble and pharmaceutically acceptable. Examples of such compounds include: sugars, sugar alcohols, sugar acids and uronic acids. Preferred polyols are sugars, sugar alcohols and sugar acids, including, but not limited to: mannitol, glycerin, xylitol, sorbitol and propylene glycol. In one embodiment, the polyol of the borate/polyol system is at least 70% by weight, more particularly at least 90% by weight, substantially entirely or entirely mannitol, sorbitol or a combination thereof.

When used, the borate/polyol complex antimicrobial buffer system (i.e., the concentration of borate added to the concentration of polyol) is typically at least 0.03 w/v %, more typically at least 0.2 w/v % and even possibly at least 0.5 w/v % of the composition, the vehicle or both. When used, the borate/polyol complex antimicrobial system is typically less than 5.0 w/v %, more typically less than 2.0 w/v % and even possibly less than 1.1 w/v % of the vehicle, the composition or both.

The compositions of the present invention will generally be formulated as sterile aqueous solutions. The compositions of the present invention are also formulated so as to be compatible with the eye and/or other tissues to be treated with the compositions. The ophthalmic compositions intended for direct application to the eye will be formulated so as to have a pH and tonicity that are compatible with the eye. It is also contemplated that the compositions can be suspensions or other types of solutions. Furthermore, the ophthalmic composition intended for direct application to the eye can be contained within an eyedropper such that a use may apply one or more drops per dose to the surface of the eyeball.

The compositions will typically have a pH in the range of 4 to 9, preferably 5.5 to 8.5, and most preferably 5.5 to 8.0. Particularly desired pH ranges are 6.0 to 7.8 and more specifically 6.2 to 7.7. The composition will also have a viscosity that is typically no greater than 150 cps, more typically no greater than 80 cps and even more typically no greater than 70 cps when viscosity of the composition is taken using a Brookfield viscometer CPE-52@60 rpm and a temperature of 25° C. Further, the composition will typically have an osmolality of at least 200 milliosmoles per kilogram (mOsm/kg), more typically at least 250 mOsm/kg and even more typically at least 275 mOsm/kg, but typically no greater than 400 mOsm/kg, more typically no greater than 350 mOsm/kg and even more typically no greater than mOsm/kg.

In addition to the ingredients above, it is contemplated that a variety of additional or alternative ingredients may be employed in the compositions or vehicles of the present invention. Other additional therapeutic agents, antimicrobials, suspension agents or the like may be included. Other exemplary ingredients possible for the composition or vehicle include, without limitation, tonicity agents, buffering agents, anti-oxidants, combinations thereof or the like. Water will make up a substantial portion of the aqueous solutions as will become apparent from the examples below. Hydrochloric acid, sodium hydroxide or other acids or bases may be used to adjust pH.

It is typically preferable for, the compositions or vehicles of the present invention to have sufficient antimicrobial activity to allow them to satisfy the certain preservative efficacy requirements, particularly USP preservative efficacy requirements and/or Ph. Eur. B and/or Ph. Eur. A.

The preservative efficacy standards for multi-dose ophthalmic solutions in the U.S. and other countries/regions are set forth in the following table:

### Preservative Efficacy Test (“PET”) Criteria (Log Order Reduction of Microbial Inoculum Over Time

| | Bacteria | Fungi |
|---|---|---|
| USP 27 | A reduction of 1 log (90%), | The compositions must |
| | by day 7; 3 logs (99.9%) | demonstrate over the |
| | by day 14; and no increase | entire test period, which |
| | after day 14 | means no increases of 0.5 |
| | | logs or greater, relative to |
| | | the initial inoculum. |
| Japan | 3 logs by 14 days; and no | No increase from initial |
| | increase from day 14 | count at 14 and 28 days |
| | through day 28 | |
| Ph. Eur. A¹ | A reduction of 2 logs (99%) | A reduction of 2 logs (99%) |
| | by 6 hours; 3 logs by 24 | by 7 days, and no increase |
| | hours; and no recovery | thereafter |
| | after 28 days | |
| Ph. Eur. B | A reduction of 1 log at 24 | A reduction of 1 log (90%) by |
| | hours; 3 logs by day 7; and | day 14, and no increase |
| | no increase thereafter | thereafter |
| FDA/ISO | A reduction of 3 logs from | No increase higher than |
| 14730 | initial challenge at day 14; | the initial value at day 14, |
| | and a reduction of 3 logs | and no increase higher than |
| | from rechallenge | the day 14 rechallenge count |
| | | through day 28 |

¹There are two preservative efficacy standards in the European Pharmacopoeia “A” and “B”.

The standards identified above for the USP 27 are substantially identical to the requirements set forth in prior editions of the USP, particularly USP 24, USP 25 and USP 26.

The following examples are presented to illustrate further various aspects of the present invention, but are not intended to limit the scope of the invention in any respect.

### EXAMPLES

Table 1 below shows the ability of PEG 6000 to solubilize Olopatadine.

TABLE I

| | | Amount | |
|---|---|---|---|
| | PEG 6000 | (mg/mL) | Viscosity |
| Sample^{a} | (%) | as Free Base | (cPs) |
| E | 0 | 2.261, 2.262, | 0.34 CPE-42 @ |
| | | 2.297, 2.288 | 30 rpm |
| F | 5 | 3.318, 3.307 | 2.10 CPE-42 @ |
| | | | 30 rpm |
| G | 10 | 4.197, 4.234 | 3.95 CPE-42 @ |
| | | | 30 rpm |
| H | 15 | 5.376, 5.395 | 6.94 CPE-42 @ |
| | | | 30 rpm |
| I | 20 | 6.293, 6.261 | 11.7 CPE-42 @ |
| | | | 30 rpm |
| J | 25 | 7.044, 7.055 | 19.4CPE-42 @ |
| | | | 30 rpm |
| K | 50 | 9.723, 9.607 | 165.9 CPE-52 @ |
| | | | 30 rpm |

^{a}Prepared in 5 mM sodium phosphate dodecahydrate. Final pH @ 7.2

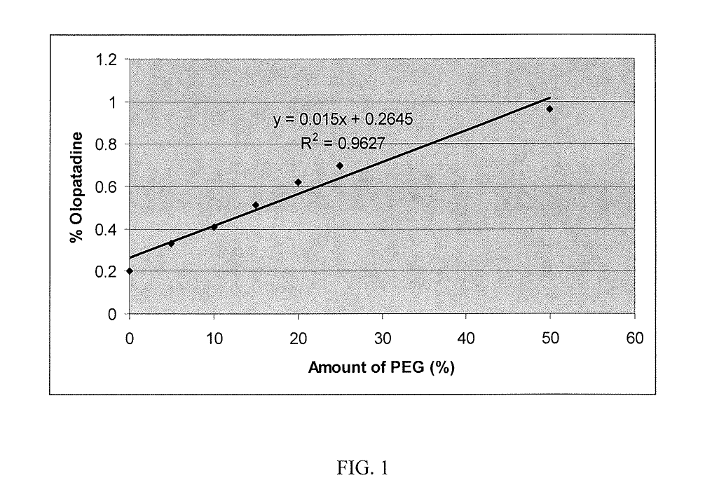

As can be seen from Table 1 and FIG. 1, the solubility of olopatadine is essentially linear relative to the concentration of PEG6000.
Table 2 below shows the ability of PVP to solubilize Olopatadine.

TABLE II

| | | Amount of | Amount | |
|---|---|---|---|---|
| | Type of | Povidone | (mg/mL) as | Viscosity |
| Sample^{a} | Povidone | (%) | Free Base | (cPs) |
| L | K-25 | 10 | 6.683 | NT |
| M | K-25 | 20 | 8.895 | 16.1 CPE-52 @ |
| | | | | 60 rpm |
| N | K-29-32 | 10 | 6.256 | NT |
| O | K-29-32 | 20 | 9.265 | 24.3 CPE-52 @ |
| | | | | 60 rpm |
| P | K-90 | 20 | 10.836 | 4607 CPE-52 @ |
| | | | | 1.5 rpm |

^{a}Prepared in borate/mannitol buffer Final pH @ 7.4
NT: Not tested

Table 3 below shows the ability of a combination of PEG 6000 and PVP to solubilize Olopatadine.

TABLE III

| | | Amount (mg/mL) |
|---|---|---|
| Sample^{a} | Description | as Free Base |
| Q | 10% PEG 6000/10% PVP K25 | 7.705, 7.794 |
| R | 10% PEG 6000/10% PVP K29/32 | 7.880, 7.885 |

^{a}Prepared in borate/mannitol buffer. Final pH @ 7.4

Table 4 below shows compositions with PEG6000 and a surfactant and particularly a tetra-functional block copolymer that is based on ethylene oxide and propylene oxide.

TABLE IV

| FID | 116351 | 116352 | 116353 | 116354 |
|---|---|---|---|---|
| Components | Amount (% w/v) | | | |
| Olopatadine | — | 0.5 | 0.75 | 1.0 |
| Hydrochloride | | | | |
| (as free base) | | | | |
| PEG 6000 | 10 | 10 | 10 | 10 |
| Tetronic 1304 | 2 | 2 | 2 | 2 |
| Dibasic sodium | 0.18 | 0.18 | 0.18 | 0.18 |
| phosphate | | | | |
| dodecahydrate | | | | |
| Mannitol | 1.7 | 1.2 | 1 | 0.75 |
| Benzalkonium | 0.01 | 0.01 | 0.01 | 0.01 |
| chloride | | | | |
| Sodium | q.s. to pH 7.2 | q.s. to pH 7.2 | q.s. to pH 7.2 | q.s. to pH 7.2 |
| hydroxide/ | | | | |
| Hydrochloric | | | | |
| acid | | | | |
| Purified water | q.s. to 100 | q.s. to 100 | q.s. to 100 | q.s. to 100 |

The formulations of Table 4 showed both early stage and late stage efficacy in animal models. These formulations were also determined to be comfortable when administered topically to the eyes as is further detailed below.
Tables 5 through 7 below illustrate examples of compositions suitable in the present invention and shows ranges for ingredients suitable for the present invention. These ranges are exemplary and not intended to be limiting unless otherwise specifically stated.

TABLE V

| Components | Amount (% w/v) |
|---|---|
| Active | 0.001-1 |
| PEG (≦10000) | 10-50 |
| Tetronic 1304 | 0-1 |
| Boric acid | 0.2-0.5 |
| Mannitol | 0.3-1.5 |
| Benzalkonium chloride | 0.005-0.01 |
| Sodium hydroxide/ | q.s. to pH 7.2 |
| Hydrochloric acid | |
| Purified water | q.s. to 100 |

TABLE VI

| Components | Amount (% w/v) |
|---|---|
| Active | 0.001-1 |
| Povidone | 10-50 |
| Tetronic 1304 | 0-1 |
| Boric acid | 0.2-0.5 |
| Mannitol | 0.2-0.5 |
| Benzalkonium chloride | 0.005-0.01 |
| Sodium hydroxide/ | q.s. to pH 7.2 |
| Hydrochloric acid | |
| Purified water | q.s. to 100 |

TABLE VII

| Components | Amount (% w/v) |
|---|---|
| Active | 0.001-1 |
| PEG (≦10000) | 10-50 |
| Boric acid | 0.2-0.5 |
| Mannitol | 0.3-1.5 |
| Propylene glycol | 0.5-1 |
| Polyquad | 0.001 |
| Sodium hydroxide/ | q.s. to pH 7.2 |
| Hydrochloric acid | |
| Purified water | q.s. to 100 |

Table 8 below provides comfort and irritation data of PEG-6000 olopatadine formulations as compared to a marketed once a day olopatadine formulation.

TABLE VIII

| | | 0.5% | 0.75% | |
|---|---|---|---|---|
| | | OLOPATADINE | OLOPATADINE | |
| | 10% | (FB) IN 10% | (FB) IN 10% | (olopatadine |
| | PEG6000- | PEG6000- | PEG6000- | hydrochloride |
| Formulation | TETRONIC- | TETRONIC- | TETRONIC- | ophthalmic solution) |
| concentration (%) | PHOS VEHICLE | PHOS VEHICLE | PHOS VEHICLE | 0.2% |
| FID | 116351 | 116352 | 116353 | NA |
| Lot No. | 09-56629-1 | 09-56630-1 | 09-56631-1 | 158586F |
| First Comfort | 1.7 ± 0.6 | 2.7 ± 0.6* | 2.0 ± 0.0 | 2.7 ± 0.6* |
| Last Comfort | 2.7 ± 0.6* | 2.3 ± 1.2* | 2.0 ± 0.0 | 2.3 ± 0.6* |
| Total Comfort | 4.3 ± 0.6 | 5.0 ± 1.0 | 4.0 ± 0.0 | 5.0 ± 0.0 |
| Score^{B} | | | | |
| Biomicroscopic | | | | |
| Evaluations^{C} | | | | |
| Conjunctival | 0.7 ± 0.6 | 0.3 ± 0.6 | 0.0 ± 0.0 | 0.3 ± 0.6 |
| Congestion | | | | |
| Conjunctival | 0.0 ± 0.0 | 0.0 ± 0.0 | 0.0 ± 0.0 | 0.0 ± 0.0 |
| Swelling | | | | |
| Conjunctival | 0.0 ± 0.0 | 0.0 ± 0.0 | 0.0 ± 0.0 | 0.0 ± 0.0 |
| Discharge | | | | |
| Conjunctival | 0.7 ± 0.6 | 0.3 ± 0.6 | 0.0 ± 0.0 | 0.3 ± 0.6 |
| Irritation^{D} | | | | |

^{A}Mean scores of three rabbits/group;
^{B}Total Comfort Score = First Comfort + Last Comfort;
^{C}No findings were noted for light reflex, flare, iritis, corneal cloudiness, fluorescein intensity, and fluorescein area during the biomicroscopic examination;
^{D}Conjunctival Irritation = (congestion) + (7*swelling) + (discharge/2).
*One or two rabbits were scored at three (eyes fully closed)

As can be seen from Table 8, the formulations of the present invention can provide a high degree of comfort while delivering high concentrations of therapeutic agent, particularly olopatadine.
Tables 9 and 10 below show that high concentration Olopatadine (0.5%) formulations according to the present invention can be preserved either by benzalkonium chloride or by polyquaternium-1. PET results of some Olopatadine formulations are provided below.

TABLE IX

| | | | |
|---|---|---|---|
| Component | | | |
| Olopatadine Hydrochloride | 0.55 | 0.55 | 0.55 |
| PEG 6000 | 10 | 10 | 10 |
| Tetronic 1304 | 2 | 2 | — |
| Povidone K29/32 | — | — | 10 |
| Dibasic Sodium Phosphate | 0.18 | — | — |
| Dodecahydrate | | | |
| Boric acid | — | 0.25 | 0.25 |
| Mannitol | — | 0.6 | 2 |
| Propylene glycol | | | |
| Benzalkonium chloride | 0.01 | — | 0.01 |
| Polyquaternium -1 | — | 0.001 | — |
| Sodium hydroxide/ | q.s. to pH | q.s. to pH | q.s. to pH |
| Hydrochloric acid | | | |
| Purified water | q.s. to 100 | q.s. to 100 | q.s. to 100 |
| Final pH | 7.2 | 7.2 | 7.4 |
| PET DATA | | | |
| S. aureus | 4.2/4.9/4.9/ | 4.9/4.9/4.9/ | 5.0/5.0/5.0/ |
| 6 h/24 h/7 d/14 d/28 d | 4.9/4.9 | 4.9/4.9 | 5.0/5.0 |
| P. aerugin | 4.9/4.9/4.9/ | 4.9/4.9/4.9/ | 5.0/5.0/5.0/ |
| 6 h/24 h/7 d/14 d/28 d | 4.9/4.9 | 4.9/4.9 | 5.0/5.0 |
| E. coli | 1.8/3.0/5.0/ | 5.0/5.0/5.0/ | 5.0/5.0/5.0/ |
| 6 h/24 h/7 d/14 d/28 d | 5.0/5.0 | 5.0/5.0 | 5.0/5.0 |
| C. albican | 3.3/4.4/4.8 | 4.8/4.8/4.8 | 4.9/4.9/ |
| 7 d/14 d/28 d | | | |
| A. niger | 0.3/1.2/1.9 | 2.1/2.1/2.1 | 5.2/5.2 |
| 7 d/14 d/28 d | | | |

TABLE X

| | | | | |
|---|---|---|---|---|
| Component | | | | |
| Olopatadine | 0.55 | 0.55 | 0.55 | 0.55 |
| Hydrochloride | | | | |
| PEG 6000 | 10 | 20 | 20 | 20 |
| Tetronic 1304 | — | — | — | — |
| Povidone K29/32 | 10 | — | — | — |
| Dibasic Sodium | — | — | — | — |
| Phosphate | | | | |
| Dodecahydrate | | | | |
| Boric acid | 0.25 | 0.25 | 0.25 | 0.25 |
| Mannitol | 2 | 2 | 2 | 0.75 |
| Propylene glycol | — | — | — | 0.5 |
| Benzalkonium | — | 0.01 | — | — |
| chloride | | | | |
| Polyquaternium -1 | 0.001 | — | 0.001 | 0.001 |
| Sodium hydroxide/ | q.s. to pH | q.s. to pH | q.s. to pH | q.s. to pH |
| Hydrochloric acid | | | | |
| Purified water | q.s. to 100 | q.s. to 100 | q.s. to 100 | q.s. to 100 |
| Final pH | 7.4 | 7.4 | 7.4 | 7.4 |
| PET DATA | | | | |
| S. aureus | 5.0/5.0/5.0/ | 5.0/5.0/5.0/ | 5.0/5.0/5.0/ | 5.0/5.0/5.0/ |
| 6 h/24 h/7 d/14 d/28 d | 5.0/5.0 | 5.0/5.0 | 5.0/5.0 | 5.0/5.0 |
| P. aerugin | 5.0/5.0/5.0/ | 5.0/5.0/5.0/ | 5.0/5.0/5.0/ | 5.0/5.0/5.0/ |
| 6 h/24 h/7 d/14 d/28 d | 5.0/5.0 | 5.0/5.0 | 5.0/5.0 | 5.0/5.0 |
| E. coli | 5.0/5.0/5.0/ | 4.9/4.9/4.9/ | 4.9/4.9/4.9/ | 4.9/4,9/4.9/ |
| 6 h/24 h/7 d/14 d/28 d | 5.0/5.0 | 4.9/4.9 | 4.9/4.9 | 4.9/4.9 |
| C. albican | 4.9/4.9/ | 4.8/4.8/ | 4.8/4.8/ | 4.8/4.8/ |
| 7 d/14 d/28 d | | | | |
| A. niger | 1.0/1.3 | 5.1/5.1 | 0.9/1.1 | 1.8/2.1 |
| 7 d/14 d/28 d | | | | |

Table 11 shows two compositions according to the present invention, the composition including high concentrations of olopatadine, high concentrations of PEG-6000 and sodium pyruvate for stabilization of the composition, particularly olopatadine.

TABLE XI

| | Concentration, | Concentration, |
|---|---|---|
| Components | % w/w | % w/w |
| Olopatadine.HCl | 0.555 | 0.777 |
| Polyethylene Glycol 6000 | 25 | 30 |
| Benzalkonium Chloride | 0.01 + 3% xs | 0.01 + 3% xs |
| Boric Acid | 0.5 | 0.5 |
| Mannitol | 0.25 | 0.25 |
| Sodium Pyruvate | 0.02 | 0.02 |
| HCl and or NaOH | QS pH 7.4 | QS pH 7.4 |

Tables 12 and 13 represent a stability study of compositions according to the present invention relative to composition D, which represents a marketed product.

TABLE XII

| | % | PEG | PVP | PVP | % PA, |
|---|---|---|---|---|---|
| Formulation | Olopatadine | 6000 | K29-32* | K29-32* | % ST, |
| ID | as F.B. | (% w/w) | (untreated) | (treated) | or % BH |
| A | 0.5 | 25 | 0 | 0 | 0 |
| B | 0.5 | 25 | 0 | 3 | 0 |
| C | 0.5 | 25 | 3 | 0 | 0 |
| C-1 | 0.5 | 25 | 3 | 0 | 0.02 ST |
| C-2 | 0.5 | 25 | 3 | 0 | 0.02 SB |
| C-3 | 0.5 | 25 | 3 | 0 | 0.02 SP |
| D | 0.2 | 0 | 0 | 1.8 | 0 |

*All formulations except D contain 0.5% borate and 0.25% mannitol and pH were adjusted to 7.4 with NaOH/HCl.
**20% PVP K29-32 aq. solution was adjusted to pH 11.5 and heated in water bath at 70-75° C. for 50 minutes.
ST = Sodium Thiosulfate;
SB = Sodium Borohydride;
SP = Sodium Pyruvate

TABLE XIII

| | | 2 weeks | | | 4 weeks | | |
|---|---|---|---|---|---|---|---|
| | | % | % N- | % | % | % N- | % |
| Formulation | Temp. | Olo. | oxide | Imp. X | Olo. | oxide | Imp. X |
| A: 0.5% Olo. + | 40° C. | 99.6 | 0 | 0 | 99.2 | 0.03 | 0 |
| 25% PEG6000 | 60° C. | 98.3 | 0.81 | 0.10 | 94.7 | 2.70 | 0.31 |
| B: 0.5% Olo. + | 40° C. | 100.3 | 0.09 | 0 | 100.0 | 0.14 | 0.01 |
| 25% PEG6000 + | 60° C. | 99.3 | 1.08 | 0.13 | 96.9 | 1.92 | 0.34 |
| 3% treated PVP | | | | | | | |
| C: 0.5% Olo. + | 40° C. | 99.5 | 0.64 | 0 | 98.9 | 0.71 | 0.02 |
| 25% PEG6000 + | 60° C. | 98.9 | 1.32 | 0.18 | 96.9 | 1.92 | 0.41 |
| 3% untreated PVP | | | | | | | |
| C-1 = C + | 40° C. | 99.7 | 0.09 | 0 | 98.1 | 0.24 | 0 |
| 0.02% Sodium | 60° C. | 99.3 | 1.07 | 0.05 | 96.4 | 2.26 | 0.08 |
| Thiosulfate | | | | | | | |
| C-2 = C + | 40° C. | 100.2 | 0 | 0 | 98.4 | 0.09 | 0.02 |
| 0.02% Sodium | 60° C. | 100.2 | 0.52 | 0.18 | 97.1 | 1.14 | 0.43 |
| Borohydride | | | | | | | |
| C-3 = C + | 40° C. | 100.5 | 0 | 0 | 98.5 | 0.02 | 0.01 |
| 0.02% Sodium | 60° C. | 100.7 | 0.06 | 0.07 | 98.2 | 0.14 | 0.14 |
| Pyruvate | | | | | | | |
| D | 40° C. | 99.6 | 0 | 0 | 99.0 | 0.06 | 0 |
| | 60° C. | 98.6 | 0.21 | 0 | 96.6 | 0.36 | 0.07 |

As can be seen, Table 13 show concentrations of impurities and N-oxide in the compositions of Table 12 when those compositions are stored at stressed conditions (i.e., elevated temperature).

We claim:

1. A multi-dose aqueous pharmaceutical composition, comprising:
therapeutic agent that exhibits relatively low solubility in water wherein the therapeutic agent is present and solubilized in the composition at a concentration that is at least 100% greater than a concentration of the therapeutic agent at a maximum solubility of the therapeutic agent in water alone;
solubility enhancing polymer present in the composition at a concentration that is at least 5 w/v % but no greater than 50 w/v % wherein the solubility enhancing polymer is selected from a polyether polymer, a polyvinyl polymer or a combination thereof; and
at least 50 w/v % water.

2. A composition as in claim 1 wherein the solubility enhancing polymer includes polyethylene glycol.

3. A composition as in claim 1 wherein the solubility enhancing polymer includes at least 90% by weight polyethylene glycol.

4. A composition as in claim 3 wherein the polyethylene glycol has a number average molecular weight that is at least 4000 but no greater than 8000.

5. A composition as in claim 4 wherein the therapeutic agent is solubilized in the composition at a concentration that is at least 200% greater than a concentration of the therapeutic agent at a maximum solubility of the therapeutic agent in water alone.

6. A composition as in claim 1 wherein the therapeutic agent is solubilized in the composition at a concentration that is at least 200% greater than a concentration of the therapeutic agent at a maximum solubility of the therapeutic agent in water alone.

7. A composition as in claim 4 further comprising a stabilizer selected from an anti-oxidant, a reducing agent or a combination thereof.

8. A composition as in claim 1 further comprising a stabilizer selected from an anti-oxidant, a reducing agent or a combination thereof.

9. A composition as in claim 7 wherein the stabilizer is selected from sodium thiosulfate, sodium borohydride, sodium pyruvate and combinations thereof.

10. A composition as in claim 8 wherein the stabilizer is selected from sodium thiosulfate, sodium borohydride, sodium pyruvate and combinations thereof.

11. A composition as in claim 1 wherein the composition satisfies Ph. Eur. A, Ph. Eur. B or both.

12. A composition as in claim 4 wherein the composition satisfies Ph. Eur. A, Ph. Eur. B or both.

13. A composition as in claim 1 wherein the composition is a multi-dose ophthalmic composition disposed within an eyedropper.

14. A composition as in any of the preceding claims wherein the composition has an osmolality of at least 200 milliosmoles per kilogram (mOsm/kg) but no greater than 400 mOsm/kg.

15. A multi-dose aqueous ophthalmic composition, comprising:
therapeutic agent that exhibits relatively low solubility in water wherein the therapeutic agent is present and solubilized in the composition at a concentration that is at least 150% greater than a concentration of the therapeutic agent at a maximum solubility of the therapeutic agent in water alone;
solubility enhancing polymer present in the composition at a concentration that is at least 10 w/v % but no greater than 50 w/v % wherein the solubility enhancing polymer includes at least 90% by weight polyethylene glycol and the polyethylene glycol has a number average molecular weight that is at least 5000 but is no greater than 7000;
an antioxidant or reducing agent selected from sodium pyruvate, sodium borohydride and sodium thiosulfate; and
at least 50 w/v % water.

16. A composition as in claim 15 wherein the composition is disposed within an eye dropper.

17. A composition as in claim 15 wherein the composition satisfies Ph. Eur. A, Ph. Eur. B or both.

18. A composition as in claim 16 wherein the composition satisfies Ph. Eur. A, Ph. Eur. B or both.

19. A method of administering an ophthalmic composition to the eye comprising:
applying the composition of claim 15 to the surface of the eyeball.

20. A method of administering an ophthalmic composition to the eye comprising:
applying the composition of claim 16 to the surface of the eyeball as one or more eyedrops from the eyedropper.
